(19) United States
(12) Patent Application Publication
(10) Pub. No.: US 2007/0275055 A1
(43) Pub. Date: Nov. 29, 2007

(54) Compositions capable of facilitating penetration across a biological barrier

(75) Inventors: Shmuel A. Ben-Sasson, Jerusalem (IL); Einat Cohen, Jerusalem (IL)

Correspondence Address: MINTZ, LEVIN, COHN, FERRIS, GLOVSKY;AND POPEO, P.C., ONE FINANCIAL CENTER, BOSTON, MA, 02111, US

(73) Assignee: Chiasma, Inc.

(21) Appl. No.: 11/879,374

(22) Filed: Jul. 16, 2007

Related U.S. Application Data
(63) Continuation of application No. 10/664,989, filed on Sep. 17, 2003, now abandoned.

(51) Int. Cl. A61K 31/727; A61K 31/728

(52) U.S. Cl. 424/452; 424/465; 514/2; 514/44; 514/54; 514/56

(57) ABSTRACT

This invention relates to novel pharmaceutical compositions mixing one or more effectors (anionic impermeable molecules) with a counter ion to the effector (a liquid forming cation). The invention also relates to methods of treating or preventing diseases by administering pharmaceutical compositions to affected subjects.

### RELATED APPLICATION

This application is a continuation application of, U.S. Ser. No. 10/664,989, filed Sep. 17, 2003; the content of which is incorporated herein by reference in its entirety.

### FIELD OF THE INVENTION

This invention relates to novel compositions capable of facilitating penetration of an effector across biological barriers utilizing ionic liquid forming cations.

### BACKGROUND OF THE INVENTION

Techniques enabling efficient transfer of a substance of interest across a biological barrier are of considerable interest in the field of biotechnology. For example, such techniques may be used for the transport of a variety of different substances across a biological barrier regulated by tight junctions (i.e., the mucosal epithelia, which includes the intestinal and respiratory epithelia and the vascular endothelia, which includes the blood-brain barrier).

The intestinal epithelium represents the major barrier to absorption of orally administered compounds, e.g., drugs and peptides, into the systemic circulation. This barrier is composed of a single layer of columnar epithelial cells (primarily enterocytes, goblet cells, endocrine cells, and paneth cells), which are joined at their apical surfaces by the tight junctions. See Madara et al., PHYSIOLOGY OF THE GASTROINTESTINAL TRACT; 2^{nd} Ed., Johnson, ed., Raven Press, New York, pp. 1251-66 (1987).

Compounds that are presented in the intestinal lumen can enter the blood stream through active or facilitative transport, passive transcellular transport, or passive paracellular transport. Active or facilitative transport occurs via cellular carriers, and is limited to transport of low molecular weight degradation products of complex molecules such as proteins and sugars, e.g., amino acids, pentoses, and hexoses. Passive transcellular transport requires partitioning of the molecule through both the apical and basolateral membranes. This process is limited to relatively small hydrophobic compounds. See Jackson, PHYSIOLOGY OF THE GASTROINTESTINAL TRACT; 2^{nd} Ed., Johnson, ed., Raven Press, New York, pp. 1597-1621 (1987). Consequently, with the exception of those molecules that are transported by active or facilitative mechanisms, absorption of larger, more hydrophilic molecules is, for the most part, limited to the paracellular pathway. However, the entry of molecules through the paracellular pathway is primarily restricted by the presence of the tight junctions. See Gumbiner, Am. J. Physiol., 253:C749-C758 (1987); Madara, J. Clin. Invest., 83:1089-94 (1989).

Considerable attention has been directed to finding ways to increase paracellular transport by “loosening” tight junctions. One approach to overcoming the restriction to paracellular transport is to co-administer, in a mixture, biologically active ingredients with absorption enhancing agents. Generally, intestinal/respiratory absorption enhancers include, but are not limited to, calcium chelators, such as citrate and ethylenediamine tetraacetic acid (EDTA); surfactants, such as sodium dodecyl sulfate, bile salts, palmitoylcarnitine, and sodium salts of fatty acids. For example, EDTA, which is known to disrupt tight junctions by chelating calcium, enhances the efficiency of gene transfer into the airway respiratory epithelium in patients with cystic fibrosis. See Wang, et al., Am. J. Respir. Cell Mol. Biol., 22:129-138 (2000). However, one drawback to all of these methods is that they facilitate the indiscriminate penetration of any nearby molecule that happens to be in the gastrointestinal or airway lumen. In addition, each of these intestinal/respiratory absorption enhancers has properties that limit their general usefulness as a means to promote absorption of various molecules across a biological barrier.

Moreover, with the use of surfactants, the potential lytic nature of these agents raises concerns regarding safety. Specifically, the intestinal and respiratory epithelia provides a barrier to the entry of toxins, bacteria and viruses from the hostile exterior. Hence, the possibility of exfoliation of the epithelium using surfactants, as well as the potential complications arising from increased epithelial repair, raise safety concerns about the use of surfactants as intestinal/respiratory absorption enhancers.

When calcium chelators are used as intestinal/respiratory absorption enhancers, Ca⁺² depletion does not act directly on the tight junction, but, rather, induces global changes in the cells, including disruption of actin filaments, disruption of adherent junctions, diminished cell adhesion, and activation of protein kinases. See Citi, J. Cell Biol., 117:169-178 (1992). Moreover, as typical calcium chelators only have access to the mucosal surface, and luminal Ca⁺² concentration may vary, sufficient amounts of chelators generally cannot be administered to lower Ca⁺² levels to induce the opening of tight junctions in a rapid, reversible, and reproducible manner.

Additionally, some toxins such as Clostridium difficile toxin A and B, appear to irreversibly increase paracellular permeability and are thus, associated with destruction of the tight junction complex. See Hecht, et al., J. Clin. Invest., 82:1516-24 (1988); Fiorentini and Thelestam, Toxicon, 29:543-67 (1991). Other toxins such as Vibrio cholerae zonula occludens toxin (ZOT) modulate the structure of intercellular tight junctions. As a result, the intestinal mucosa becomes more permeable. See Fasano, et al., Proc. Nat. Acad. Sci., USA, 8:5242-46 (1991); U.S. Pat. No. 5,827,534. However, this also results in diarrhea.

Therefore, large hydrophilic molecules of therapeutic value present a difficult problem in the field of drug delivery. While they are readily soluble in water, and thus easily dissolve in physiological media, such molecules are barred from absorption by the mucosal layer due to their cell membrane impermeability. The epithelial cell membrane is composed of a phospholipid bilayer in which proteins are embedded via hydrophobic segments. Thus, the cell membrane constitutes a very strong barrier for transport of hydrophilic substances, including peptides and proteins.

Several new methods for the delivery of proteins across cell membranes are being evaluated, although these are still lacking in convenience and effectiveness. The most popular method utilizes “protein transduction domains” or “membrane transport signals”. These are derived from viral proteins, or synthetically from phage display libraries, and are characterized by a high content of positively charged lysine and arginine residues. See Schwarze, et al., Science, 285:1569-1572 (1999); Rojas, et al., Nat. Biotechnol., 16:370-375 (1998). Microinjection and electroporation techniques have also been utilized with varying degrees of success.

Lately, alternative methods using a cationic lipid formulation have been suggested. See Zelphati, et al., J. Biol. Chem., 276: 35103-35110, who utilize trifluoroacetylated lipopolyamine and dioleoyl phosphatidylethanolamine, for the delivery of proteins and peptides into the cytoplasm. See also the use of lipoamino acid conjugates and liposaccharide conjugates by Toth, et al., J. Drug Targeting, 2:217-239 (1994), and proceedings thereof. These methods all utilize amphipathic molecules which bind, covalently or otherwise, the target molecule, thus “hydrophobizing” its original charge and enabling its penetration through the lipophylic cell membrane.

The use of amphipathic counter ions shows promise for an efficient, non-invasive, low-risk means for the delivery of biologically active molecules, such as polypeptides, drugs and other therapeutic agents, across various biological barriers.

### SUMMARY OF THE INVENTION

The present invention provides compositions for translocating therapeutically active anionic impermeable molecules. The invention also relates to methods of using a counter ion to the effector to translocate at least one effector across a biological barrier. The counter ion can be an ionic liquid forming cation, which can include a hydrophobic moiety. In one preferred embodiment, the counter ion is an ionic liquid forming cation. As used herein, those skilled in the art will recognize that all references to counter ions herein are meant to encompass this preferred embodiment. Specifically, the invention involves a hydrophobic composition having a therapeutically effective amount of at least one effector, and a counter ion to the at least one effector in order to enable the effector's translocation across a biological barrier. The action of the cation can be modified by addition of hydrophobic moieties. A hydrophobic agent can be a single molecule or a combination of hydrophobic molecules, like aliphatic or aromatic molecules. Examples of aliphatic hydrophobic agents include fatty acids, mono-, di-, or tri-glycerides, ethers, or cholesterol esters of fatty acids. The tri-glyceride can be tricaprin, for example. An example of an aromatic hydrophobic agent includes benzyl benzoate.

As used herein a “hydrophobic composition” includes any composition that is water insoluble and facilitates the translocation of a substance, e.g., at least one effector, across a biological barrier utilizing at least one counter ion (i.e., an ionic liquid forming cationic counter ion as described herein). As used herein, the term “biological barrier” is meant to include biological membranes such as the plasma membrane as well as any biological structures sealed by tight junctions (or occluding junctions) such as the mucosal or vascular epithelia, including, but not limited to, the intestinal or respiratory epithelia, and the blood brain barrier. Moreover, those skilled in the art will recognize that translocation may occur across a biological barrier in a tissue such as epithelial cells or endothelial cells.

The invention also provides hydrophobic compositions having a pharmaceutically acceptable carrier or excipient, or a combination thereof. In various embodiments, the compositions of the invention can be contained within a capsule, or can take the form of a tablet, an aqueous dispersion, suspension, or emulsion, a cream, an ointment, or a suppository. Likewise, the composition can be dissolved in an at least partially water soluble solvents, such as, for example, alcohols, (e.g., n-butanol, isoamyl (=isopentyl) alchohol, iso-butanol, iso-propanol, propanol, ethanol, ter-butanol), polyols, DMF, DMSO, ethers, amides, esters, or various mixtures thereof.

Hydrophobic compositions can include at least one effector coupled to a suitable counter ion. The at least one effector can be a therapeutically active anionic impermeable molecule including, but not limited to, nucleic acids, glycosaminoglycans, proteins, peptides, or pharmaceutically active agents, such as, for example, hormones, growth factors, neurotrophic factors, anticoagulants, bioactive molecules, toxins, antibiotics, anti-fungal agents, antipathogenic agents, antigens, antibodies, antibody fragments, immunomodulators, vitamins, antineoplastic agents, enzymes, or therapeutic agents. For example, glycosaminoglycans acting as anionic impermeable compounds include, but are not limited to, heparin, heparan sulfate, chondroitin sulfate, dermatan sulfate, and hyaluronic acid. Nucleic acids serving as anionic impermeable molecules include, but are not limited to, specific DNA sequences (e.g., coding genes), specific RNA sequences (e.g., RNA aptamers, antisense RNA or a specific inhibitory RNA (RNAi)), poly CpG, or poly I:C synthetic polymers of nucleic acids. Other suitable proteins include, but are not limited to, hormones, gonadotropins, growth factors, cytokines, neurotrophic factors, immunomodulators, enzymes, anticoagulants, toxins, antigens, antipathogenic agents, antineoplastic agents, antibodies, antibody fragments, and other therapeutic agents. Specifically these include, but are not limited to, insulin, erythropoietin (EPO), glucagon-like peptide 1 (GLP-1), αCMSH, parathyroid hormone (PTH), growth hormone, calcitonin, interleukin-2 (IL-2), α1-antitrypsin, granulocyte/monocyte colony stimulating factor (GM-CSF), granulocyte colony stimulating factor (G-CSF), T20, anti-TNF antibodies, interferon α, interferon β, interferon γ, lutenizing hormone (LH), follicle-stimulating hormone (FSH), enkephalin, dalargin, kyotorphin, basic fibroblast growth factor (bFGF), hirudin, hirulog, lutenizing hormone releasing hormone (LHRH) analog, brain-derived natriuretic peptide (BNP), and neurotrophic factors.

As used herein, “anionic impermeable molecules” are molecules that are negatively charged and are unable to efficiently cross biological barriers, such as the cell membrane or tight junctions. Preferably, anionic impermeable molecules of the invention are of a molecular weight above 200 daltons. Anionic impermeable molecules are preferably polysaccharides, i.e., glycosaminoglycans, nucleic acids or net negatively charged proteins. A protein's net charge is determined by two factors: 1) the total count of acidic amino acids vs. basic amino acids, and 2) the specific solvent pH surroundings, which expose positive or negative residues. As used herein, “net negatively charged proteins” are proteins that, under non-denaturing pH surroundings, have a net negative electric charge. For example, insulin is a 51 amino acid protein that contains two positively charged residues, one lysine and one arginine, and four glutamic acid residues. Therefore, under neutral or basic pH surroundings, insulin constitutes a net negatively charged protein. In general, those skilled in the art will recognize that all proteins may be considered “net negatively charged proteins”, regardless of their amino acid composition, depending on their pH and/or solvent surroundings. For example, different solvents can expose negative or positive side chains depending on the solvent pH.

Hydrophobic compositions according to the invention can also be used to enhance the penetration of smaller molecules that are otherwise impermeable through epithelial barriers. Examples of such molecules include nucleic acids (i.e., DNA, RNA, or mimetics thereof).

Counter ions of this invention can include, for example, cationic amphipathic molecules. In one embodiment, cationic counter ions of this invention are ions that are positively charged and can include a hydrophobic moiety. Under appropriate conditions, cationic counter ions can establish electrostatic interactions with anionic impermeable molecules. The formation of such a complex can cause charge neutralization, thereby creating a new uncharged entity, with further hydrophobic properties due to the inherent hydrophobicity of the counter ion.

Contemplated cationic counter ions can include ionic liquid forming cations, such as imidazolium derivatives, pyridinium derivatives, phosphonium compounds or tetralkylammonium compounds. For example, imidazolium derivatives have the general structure of 1-R1-3-R2-imidazolium where R1 and R2 can be linear or branched alkyls with 1 to 12 carbons. Such imidazolium derivatives can be further substituted for example by halogens or an alkyl group. Specific imidazolium derivatives include, but are not limited to, 1-ethyl-3-methylimidazolium, 1-butyl-3-methylimidazolium, 1-hexyl-3-methylimidazolium, 1-methyl-3-octylimidazolium, 1-methyl-3-(3,3,4,4,5,5,6,6,7,7,8,8,8-tridecafluoroctyl)-imidazolium, 1,3-dimethylimidazolium, and 1,2-dimethyl-3-propylimidazolium.

Pyridinium derivatives have the general structure of 1-R1-3-R2-pyridinium where R1 is a linear or branched alkyl with 1 to 12 carbons, and R2 is H or a linear or branched alkyl with 1 to 12 carbons. Such pyridinium derivatives can be further substituted for example by halogens or an alkyl group. Pyridinium derivatives include, but are not limited to, 3-methyl-1-propylpyridinium, 1-butyl-3-methylpyridinium, and 1-butyl-4-methylpyridinium.

The invention also involves methods of translocating at least one effector across a biological barrier by using the compositions of the invention. For example, at least one effector can be coupled to a counter ion to form a composition according to the invention, which can then be introduced to a biological barrier, thereby effectively translocating the effector across the biological membrane. The counter ion can further include a hydrophobic moiety. As used herein, the term “coupled” is meant to include all such specific interactions that result in two or more molecules showing a preference for one another relative to some third molecule, including any type of interaction enabling a physical association between an effector and an ionic liquid forming cation. Preferably this includes, but is not limited to, electrostatic interactions, hydrophobic interactions and hydrogen bonding, but does not include non-specific associations such as solvent preferences. The association must be sufficiently strong so that the effector does not dissociate before or during penetration of the biological barrier.

Preferred compositions include, e.g., enteric-coated tablets and gelatin capsules comprising the active ingredient together with a) diluents, e.g., lactose, dextrose, sucrose, mannitol, sorbitol, cellulose and/or glycine; b) protease inhibitors such as Aprotinin or trasylol; c) lubricants, e.g., silica, talcum, stearic acid, its magnesium or calcium salt, poloxamer and/or polyethyleneglycol; for tablets also d) binders, e.g., magnesium aluminum silicate, starch paste, gelatin, tragacanth, methylcellulose, sodium carboxymethylcellulose and/or polyvinylpyrrolidone; e) ionic surface active agents such as poloxamer, Solutol HS15, Cremophore, phospholipids and bile acids, if desired f) disintegrants, e.g., starches, agar, alginic acid or its sodium salt, or effervescent mixtures; and/or g) absorbents, colorants, flavors and sweeteners. Suppositories are advantageously prepared from fatty emulsions or suspensions. The compositions may be sterilized and/or contain adjuvants, such as preserving, reducing agents e.g., NAC (N-Acetyl-L-Cysteine), stabilizing, wetting or emulsifying agents, solution promoters, salts for regulating the osmotic pressure and/or buffers. In addition, they may also contain other therapeutically valuable substances. The compositions are prepared according to conventional mixing, granulating or coating methods, respectively, and contain about 0.01 to 75%, preferably about 0.1 to 10%, of the active ingredient.

These compositions may further contain a mixture of at least two substances selected from the group consisting of a non-ionic detergent, an ionic detergent, a protease inhibitor, and a reducing agent. For example, the non-ionic detergent may be a poloxamer or Solutol HS 15; the poloxamer may be pluronic F-68; the ionic detergent may be a bile salt; and the bile salt may be Taurodeoxycholate; the protease inhibitor may be selected from the group consisting of aprotonin and soy bean trypsin inhibitor; and/or the reducing agent may be NAC.

Other suitable protease inhibitors that can be added to the penetration composition are described in Bernkop-Schnurch et al., J. Control. Release, 52:1-16 (1998). These include, e.g., inhibitors of luminally secreted proteases, examples of which are aprotinin, Bowman-Birk inhibitor, soybean trypsin inhibitor, chicken ovomucoid, chicken ovoinhibitor, human pancreatic trypsin inhibitor, camostate mesilate, flavonoid inhibitors, antipain, leupeptin, p-aminobenzamidine, AEBSF, TLCK, APMSF, DFP, PMSF, poly(acrylate) derivatives, chymostatin, benzyloxycarbonyl-Pro-Phe-CHO, FK-448, sugar biphenylboronic acids complexes, β-phenylpropionate, elastatinal, methoxysuccinyl-Ala-Ala-Pro-Val-chloromethylketone (MPCMK), EDTA, and chitosan-EDTA conjugates. These also include inhibitors of membrane bound proteases, such as amino acids, di- and tripeptides, amastatin, bestatin, puromycin, bacitracin, phosphinic acid dipeptide analogues, α-aminoboronic acid derivatives, Na-glycocholate, 1,10-phenantroline, acivicin, L-serine-borate, thiorphan, and phosphoramidon.

The invention also provides kits having one or more containers containing a therapeutically or prophylactically effective amount of a composition of the invention.

Also described are methods of treating or preventing a disease or pathological condition by administering to a subject in which such treatment or prevention is desired, a composition of the invention in an amount sufficient to treat or prevent the disease or pathological condition. For example, the disease or condition to be treated may include but are not limited to endocrine disorders, including diabetes, infertility, hormone deficiencies and osteoporosis; ophthalmological disorders; neurodegenerative disorders, including Alzheimer's disease and other forms of dementia, Parkinson's disease, multiple sclerosis, and Huntington's disease; cardiovascular disorders, including atherosclerosis, hyper- and hypocoagulable states, coronary disease, and cerebrovascular events; metabolic disorders, including obesity and vitamin deficiencies; renal disorders, including renal failure; haematological disorders, including anemia of different entities; immunologic and rheumatologic disorders, including autoimmune diseases, and immune deficiencies; infectious diseases, including viral, bacterial, fungal and parasitic infections; neoplastic diseases; and multi-factorial disorders, including impotence, chronic pain, depression, different fibrosis states, and short stature.

Administration of the active compounds and salts described herein can be via any of the accepted modes of administration for therapeutic agents. These methods include oral, bucal, anal, bronchial, nasal, sublingual, parenteral, transdermal, or topical administration modes.

Also included in the invention are methods of producing the compositions described herein. For example, the effector and the counter ion can be lyophilized or freeze dried together and then reconstituted under preferred solvent surroundings. The composition can be further supplemented by a polyanionic molecule, such as phytic acid, and/or any other constituent of the pharmaceutical excipient or carrier, which can be optionally added with the effector and counter ion during the lyophilization. Other components of the composition can also be optionally added during reconstitution of the lyophilized materials. Such optional components can include, for example, pluronic F-68, Aprotinin, Solutol HS-15, N-Acetyl Cysteine, and/or Tricaprin.

The effectors of the invention can also be further chemically modified. For example, one or more polyethylene glycol (PEG) residues can be attached to the therapeutic effectors of the invention.

Also provided are methods of oral or nasal, i.e., mucosal, vaccination involving administering to a subject in need of vaccination an effective amount of a composition of the invention, wherein the effector includes an antigen to which vaccination is desired. In one embodiment, the effector can be a protective antigen (PA) for use in a vaccine against Anthrax. In another embodiment, the effector can be a Hepatitis B surface antigen (HBs) for use in a vaccine against Hepatitis B.

The details of one or more embodiments of the invention have been set forth in the accompanying description below. Although any methods and materials similar or equivalent to those described herein can be used in the practice or testing of the present invention, the preferred methods and materials are now described. Other features, objects, and advantages of the invention will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this invention belongs. All patents and publications cited in this specification are incorporated by reference.

### DETAILED DESCRIPTION OF THE INVENTION

As described herein, ionic liquid forming cations can be utilized as cationic counter ions for enabling or facilitating translocation across biological barriers. Cationic counter ions of this invention are ions that are positively charged and include a hydrophobic moiety. Under appropriate conditions, cationic counter ions can establish electrostatic interactions with anionic impermeable molecules. The formation of such a complex can cause charge neutralization, thereby creating a new uncharged entity, with further hydrophobic properties due to the inherent hydrophobicity of the counter cation.

The use of the effector-counter ion hydrophobic compositions described herein allows for low immunogenicity, high reproducibility, extensive and simple application for a wide variety of therapeutic molecules, and allows for the potential for highly efficient delivery through biological barriers in an organism. Accordingly, these compositions have the potential to improve upon conventional transporters such as liposomes or viruses for the efficient delivery of many macromolecules. The methods of the present invention employ the use of an effector-counter cation complexes to create hydrophobic compositions to specifically transport macromolecules across biological barriers sealed by tight junctions.

The present invention provides compositions for penetration that specifically targets various tissues, especially epithelial and endothelial, for the delivery of drugs and other therapeutic agents across a biological barrier. Existing transport systems known in the art are too limited to be of general application because they are inefficient, they alter the biological properties of the active substance, they kill the target cell, they irreversibly destroy the biological barrier and/or they pose too high of a risk to be used in human subjects.

The compositions of the present invention exhibit efficient, non-invasive delivery of an unaltered biologically active substance, and thus, have many uses. For example, the compositions of the invention can be used in the treatment of diabetes. Insulin levels in the blood stream must be tightly regulated. The compositions of the invention can be used to deliver insulin, for example, across the mucosal epithelia at high yield. Alternative non-invasive insulin delivery methods, previously known in the art, have typical yields of 1-5% and cause intolerable fluctuations in the amount of insulin absorbed. Another treatment for elevated blood glucose levels involves the use of glucagon-like peptide 1. GLP-1 is a potent hormone, which is endogenously secreted in the gastrointestinal tract upon food injection. GLP-1's important physiological action is to augment the secretion of insulin in a glucose-dependant manner, thus allowing for treatment of diabetic states.

In addition, these compositions also can be used to treat conditions resulting from atherosclerosis and the formation of thrombi and emboli such as myocardial infarction and cerebrovascular accidents. Specifically, the compositions can be used to deliver heparin across the mucosal epithelia. Heparin is an established effective and safe anticoagulant. However, its therapeutic use is limited by the need for parenteral administration. Thus far, there has been limited success in the direction of increasing heparin absorption from the intestines, and a sustained systemic anticoagulant effect has not been achieved.

The compositions of this invention can also be used to treat hematological diseases and deficiency states that are amenable to administration of hematological growth factors. For Example, erythropoietin is a glycoprotein which stimulates red blood cell production. It is produced in the kidney and stimulates the division and differentiation of committed erythroid progenitors in the bone marrow. Endogenously, hypoxia and anemia generally increase the production of erythropoietin, which in turn stimulates erythropoiesis. However, in patients with chronic renal failure (CRF), production of erythropoietin is impaired. This erythropoietin deficiency is the primary cause of their anemia. Recombinant EPO stimulates erythropoiesis in anemic patients with CRF, including patients on dialysis, as well as those who do not require regular dialysis. Additional anemia states treated by EPO include Zidovudine-treated HIV-infected patients, cancer patients on chemotherapy. Anemia observed in cancer patients may be related to the disease itself or the effect of concomitantly administered chemotherapeutic agents.

Similarly, colony stimulating factors are also glycoproteins which act on hematopoietic cells by binding to specific cell surface receptors and stimulating proliferation, differentiation, commitment, and some end-cell functional activation. Granulocyte-colony stimulation factor (G-CSF) regulates the production of neutrophils within the bone marrow and affects neutrophil progenitor proliferation, differentiation and selected end-cell functional activation, including enhanced phagocytic ability, priming of the cellular metabolism associated with respiratory burst, antibody dependent killing, and the increased expression of some functions associated with cell surface antigens. In cancer patients, recombinant granulocyte-colony stimulating factor has been shown to be safe and effective in accelerating the recovery of neutrophil counts following a variety of chemotherapy regimens, thus preventing hazardous infectious. G-CSF can also shorten bone marrow recovery when administered after bone marrow transplantations.

The composition of this invention can also be used to administer monoclonal antibodies for different indications. For example, administration of antibodies that block the signal of tumor necrosis factor (TNF) can be used to treat pathologic inflammatory processes such as rheumatoid arthritis (RA), polyarticular-course juvenile rheumatoid arthritis (JRA), as well as the resulting joint pathology.

Additionally, the compositions of this invention can be used to treat osteoporosis. It has recently been shown that intermittent exposure to parathyroid hormone (PTH), as occurs in recombinant PTH injections, results in an anabolic response, rather than the well known catabolic reaction induced by sustained exposure to elevated PTH levels, as seen in hyperparathyroidism. Thus, non invasive administration of PTH may be beneficial for increasing bone mass in various deficiency states, including osteoporosis. See Fox, Curr. Opin. Pharmacol., 2:338-344 (2002).

Currently, the delivery of effectors (e.g., the delivery of insulin, erythropoietin, or heparin to the blood stream) requires invasive techniques such as intravenous or intramuscular injections. One advantage of the compositions of this invention is that they can deliver such effectors across biological barriers through non-invasive administration, including, for example oral, bucal, rectal, inhalation, insufflation, transdermal, or depository. In addition, a further advantage of the compositions of the invention is that they are able to cross the blood-brain barrier, thereby delivering effectors to the central nervous system (CNS).

Compositions of this invention facilitate the passage, translocation, or penetration of a substance across a biological barrier, particularly through or between cells “sealed” by tight junctions. Translocation may be detected by any method known to those skilled in the art, including using imaging compounds such as radioactive tagging, and/or fluorescent probes or dyes incorporated into a hydrophobic composition in conjunction with a paracytosis assay as described in, for example, Schilfgaarde, et al., Infect. and Immun., 68(8):4616-23 (2000). Generally, a paracytosis assay is performed by: a) incubating a cell layer with a composition described by this invention; b) making cross sections of the cell layers; and c) detecting the presence of the effectors, counter cations or compositions of this invention. The detection step may be carried out by incubating the fixed cell sections with labeled antibodies directed to a component of the compositions of this invention, followed by detection of an immunological reaction between the component and the labeled antibody. Alternatively, a component of the compositions may be labeled using a radioactive label, or a fluorescent label, or a dye in order to directly detect the presence of the component. Further, a bioassay can be used to monitor the compositions' translocation. For example, using a bioactive molecule such as insulin, included in a composition, the drop in blood glucose level can be measured.

As used herein, the term “effector” refers to any anionic impermeable molecule or compound of, for example, biological, therapeutic, pharmaceutical, diagnostic, or tracing significance. An anionic impermeable molecule can consist of nucleic acids (ribonucleic acid, deoxyribonucleic acid) from various origins, (particularly of human, viral, animal, eukaryotic or prokaryotic, plant, synthetic origin, etc). A nucleic acid of interest may be of a variety of sizes, ranging from, for example, a simple trace nucleotide to a genome fragment, or an entire genome. It may be a viral genome or a plasmid. Alternatively, the effector of interest can also be a protein, such as, for example, an enzyme, a hormone, a cytokine, an apolipoprotein, a growth factor, a bioactive molecule, an antigen, or an antibody, etc. As used herein, the term “bioactive molecule” refers to those compounds that have an effect on or elicit a response from living cells or tissues. A non-limiting example of a bioactive molecule is a protein. Other examples of the bioactive molecule include, but are not limited to, insulin, erythropoietin (EPO), glucagon-like peptide 1 (GLP-1), αMSH, parathyroid hormone (PTH), growth hormone, calcitonin, interleukin-2 (IL-2), α1-antitrypsin, granulocyte/monocyte colony stimulating factor (GM-CSF), granulocyte colony stimulating factor (G-CSF), T20, anti-TNF antibodies, interferon α, interferon β, interferon γ, lutenizing hormone (LH), follicle-stimulating hormone (FSH), enkephalin, dalargin, kyotorphin, basic fibroblast growth factor (bFGF), hirudin, hirulog, lutenizing hormone releasing hormone (LHRH) analog, brain-derived natriuretic peptide (BNP), or neurotrophic factors. The effector of interest can also be a glycosaminoglycan including, but not limited to, heparin, heparan sulfate, chondroitin sulfate, dermatan sulfate, and hyaluronic acid. The effector of interest can further be a nucleic acid such as DNA or RNA. Additionally, the effector can be a pharmaceutically active agent, such as, for example, a toxin, a therapeutic agent, or an antipathogenic agent, such as an antibiotic, an antiviral, an antifungal, or an anti-parasitic agent. The effector of interest can itself be directly active or can be activated in situ by the composition, by a distinct substance, or by environmental conditions.

The terms “pharmaceutically active agent” and “therapeutic agent” are used interchangeably herein to refer to a chemical material or compound, which, when administered to an organism, induces a detectable pharmacologic and/or physiologic effect.

The hydrophobic compositions according to the present invention are characterized by the fact that their penetration capacity is virtually independent of the nature of the effector that is included in it.

“Counter ions” according to this invention can include, for example, cationic amphipathic molecules, i.e., those having both polar and nonpolar domains, or both hydrophilic and hydrophobic properties. Cationic counter ions of this invention are ions that are positively charged and can include a hydrophobic moiety. Under appropriate conditions, cationic counter ions can establish electrostatic interactions with anionic impermeable molecules. The formation of such a complex can cause charge neutralization, thereby creating a new uncharged entity, with further hydrophobic properties due to the inherent hydrophobicity of the counter ion. In one preferred embodiment, the counter ion can be an ionic liquid forming cation.

Ionic liquids are salts composed of cations such as imidazolium ions, pyridinium ions and anions such as BF₄⁻, PF₆⁻ and are liquid at relatively low temperatures. Ionic liquids are characteristically in liquid state over extended temperature ranges, and have high ionic conductivity. Other favorable characteristic properties of the ionic liquids include non-flammability, high thermal stability, relatively low viscosity, and essentially no vapor pressure. When an ionic liquid is used as a reaction solvent, the solute is solvated by ions only, thus creating a totally different environment from that when water or ordinary organic solvents are used. This enables high selectivity, applications of which are steadily expanding. Some examples are in the Friedel-Crafts reaction, Diels-Alder reaction, metal catalyzed asymmetric synthesis and others. Furthermore, some ionic liquids have low solubility in water and low polar organic solvents, enabling their recovery after reaction product is extracted with organic solvents. Ionic liquids are also used electrochemically, due to their high ion-conductivity, for example as electrolytes of rechargeable batteries.

Contemplated cationic counter ions can be ionic liquid forming cations, such as imidazolium derivatives, pyridinium derivatives, phosphonium compounds or tetralkylammonium compounds. For example, imidazolium derivatives have the general structure of 1-R1-3-R2-imidazolium where R1 and R2 can be linear or branched alkyls with 1 to 12 carbons. Such imidazolium derivatives can be further substituted for example by halogens or an alkyl group. Specific imidazolium derivatives include, but are not limited to, 1-ethyl-3-methylimidazolium, 1-butyl-3-methylimidazolium, 1-hexyl-3-methylimidazolium, 1-methyl-3-octylimidazolium, 1-methyl-3-(3,3,4,4,5,5,6,6,7,7,8,8,8-tridecafluoroctyl)-imidazolium, 1,3-dimethylimidazolium, and 1,2-dimethyl-3-propylimidazolium.

Pyridinium derivatives have the general structure of 1-R1-3-R2-pyridinium where R1 is a linear or branched alkyl with 1 to 12 carbons, and R2 is H or a linear or branched alkyl with 1 to 12 carbons. Such pyridinium derivatives can be further substituted for example by halogens or an alkyl group. Pyridinium derivatives include, but are not limited to, 3-methyl-1-propylpyridinium, 1-butyl-3-methylpyridinium, and 1-butyl-4-methylpyridinium.

In one embodiment, the present invention relates to the use of the cationic component of ionic liquids. Unlike other ionic liquids, the salts of the cations according to the present invention are typically water soluble. For example, an anionic counterpart of the ionic liquid forming cation can be a halogen, such as chloride or bromide. Also included in the invention are methods of producing the compositions described herein. For example, the effector and the counter ion can be lyophilized or freeze dried together and then reconstituted under preferred solvent surroundings. Phytic acid and/or any other constituent of the pharmaceutical excipient or carrier can be optionally added with the effector and counter ion during the lyophilization. Other components of the composition can also be optionally added during reconstitution of the lyophilized materials. Such optional components can include, for example, pluronic F-68, Aprotinin, Solutol HS-15, N-Acetyl Cysteine, and/or Tricaprin.

It is well known to those skilled in the art that proteins can be further chemically modified to enhance the protein half-life in circulation. By way of non-limiting example, polyethylene glycol (PEG) residues can be attached to the effectors of the invention. Conjugating biomolecules with PEG, a process known as pegylation, is an established method for increasing the circulating half-life of proteins. Polyethylene glycols are nontoxic water-soluble polymers that, because of their large hydrodynamic volume, create a shield around the pegylated molecule, thereby protecting it from renal clearance, enzymatic degradation, as well as recognition by cells of the immune system.

Agent-specific pegylation methods have been used in recent years to produce pegylated molecules (e.g., drugs, proteins, agents, enzymes, etc.) that have biological activity that is the same as, or greater than, that of the “parent” molecule. These agents have distinct in vivo pharmacokinetic and pharmacodynamic properties, as exemplified by the self-regulated clearance of pegfilgrastim, the prolonged absorption half-life of pegylated interferon alpha-2a. Pegylated molecules have dosing schedules that are more convenient and more acceptable to patients, which can have a beneficial effect on the quality of life of patients. (See e.g., Yowell S. L. et al., Cancer Treat Rev 28 Suppl. A:3-6 (April 2002)).

The invention also includes methods of contacting biological barriers with compositions of the invention in an amount sufficient to enable efficient penetration of the compositions through the barrier. The hydrophobic composition of this invention can be provided in vitro, ex vivo, or in vivo. Furthermore, the compositions according to this invention may be capable of potentializing the biological activity of the included substance. Therefore, another purpose of this invention is a method of using compositions to increase the biological activity of the effector.

In addition to the hydrophobic composition, the invention also provides a pharmaceutically acceptable base or acid addition salt, hydrate, ester, solvate, prodrug, metabolite, stereoisomer, or mixture thereof. The invention also includes pharmaceutical formulations comprising hydrophobic compositions in association with a pharmaceutically acceptable carrier, diluent, protease inhibitor, surface active agent, or excipient. A surface active agent can include, for example, poloxamers, Solutol HS15, cremophore, phospholipids, or bile acids/salts

Salts encompassed within the term “pharmaceutically acceptable salts” refer to non-toxic salts of the compounds of this invention which are generally prepared by reacting the free base with a suitable organic or inorganic acid or solvent to produce “pharmaceutically-acceptable acid addition salts” of the compounds described herein. These compounds retain the biological effectiveness and properties of the free bases. Representative examples of such salts include the water-soluble and water-insoluble salts, such as the acetate, amsonate (4,4-diaminostilbene-2,2′-disulfonate), benzenesulfonate, benzoate, bicarbonate, bisulfate, bitartrate, borate, bromide, butyrate, calcium edetate, camsylate, carbonate, chloride, citrate, clavulariate, dihydrochloride, edetate, edisylate, estolate, esylate, fumarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methylene-bis-2-hydroxy-3-naphthoate, embonate), pantothenate, phosphate/diphosphate, picrate, polygalacturonate, propionate, p-toluenesulfonate, salicylate, stearate, subacetate, succinate, sulfate, sulfosaliculate, suramate, tannate, tartrate, teoclate, tosylate, triethiodide, and valerate salts.

According to the methods of the invention, a patient, i.e., a human, can be treated with a pharmacologically or therapeutically effective amount of a hydrophobic composition. As used herein the term “pharmacologically or therapeutically effective amount” means that amount of a drug or pharmaceutical agent (the effector) that will elicit the biological or medical response of a tissue, system, animal or human that is being sought by a researcher or clinician.

The invention also includes pharmaceutical compositions suitable for introducing an effector of interest across a biological barrier. The compositions are preferably suitable for internal use and include an effective amount of a pharmacologically active compound of the invention, alone or in combination, with one or more pharmaceutically acceptable carriers. The compounds are especially useful in that they have very low, if any, toxicity.

Preferred pharmaceutical compositions are tablets and gelatin capsules, enteric coated, comprising the active ingredient together with a) diluents, e.g., lactose, dextrose, sucrose, mannitol, sorbitol, cellulose and/or glycine; b) protease inhibitors including, but not limited to, aprotinin, Bowman-Birk inhibitor, soybean trypsin inhibitor, chicken ovomucoid, chicken ovoinhibitor, human pancreatic trypsin inhibitor, camostate mesilate, flavonoid inhibitors, antipain, leupeptin, p-aminobenzamidine, AEBSF, TLCK, APMSF, DFP, PMSF, poly(acrylate)derivatives, chymostatin, benzyloxycarbonyl-Pro-Phe-CHO; FK-448, sugar biphenylboronic acids complexes, β-phenylpropionate, elastatinal, methoxysuccinyl-Ala-Ala-Pro-Val-chloromethylketone (MPCMK), EDTA, chitosan-EDTA conjugates, amino acids, di-peptides, tripeptides, amastatin, bestatin, puromycin, bacitracin, phosphinic acid dipeptide analogues, α-aminoboronic acid derivatives, Na-glycocholate, 1,10-phenantroline, acivicin, L-serine-borate, thiorphan, and phosphoramidon; c) lubricants, e.g., silica, talcum, stearic acid, its magnesium or calcium salt, poloxamer and/or polyethyleneglycol; for tablets also d) binders, e.g., magnesium aluminum silicate, starch paste, gelatin, tragacanth, methylcellulose, sodium carboxymethylcellulose and/or polyvinylpyrrolidone; if desired e) disintegrants, e.g., starches, agar, alginic acid or its sodium salt, or effervescent mixtures; and/or f) absorbents, colorants, flavors and sweeteners. The compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. The compositions are prepared according to conventional mixing, granulating or coating methods, respectively, and contain about 0.01 to 75%, preferably about 0.1 to 10%, of the active ingredient.

Administration of the active compounds and salts described herein can be via any of the accepted modes of administration for therapeutic agents. These methods include oral, bucal, anal, bronchial, nasal, sublingual, parenteral, transdermal, or topical administration modes. As used herein “parenteral” refers to injections given through some other route than the alimentary canal, such as subcutaneously, intramuscularly, intraorbitally (i.e., into the eye socket or behind the eyeball), intracapsularly, intraspinally, intrasternally, or intravenously.

Depending on the intended mode of administration, the compositions may be in solid, semi-solid or liquid dosage form, such as, for example, tablets, suppositories, pills, time-release capsules, powders, liquids, suspensions, aerosol or the like, preferably in unit dosages. The compositions will include an effective amount of active compound or the pharmaceutically acceptable salt thereof, and in addition, may also include any conventional pharmaceutical excipients and other medicinal or pharmaceutical drugs or agents, carriers, adjuvants, diluents, protease inhibitors, etc., as are customarily used in the pharmaceutical sciences.

For solid compositions, excipients include pharmaceutical grades of mannitol, lactose, starch, magnesium stearate, sodium saccharin, talcum, cellulose, glucose, sucrose, magnesium carbonate, and the like may be used. The active compound defined above, may be also formulated as suppositories using for example, polyalkylene glycols, for example, propylene glycol, as the carrier.

Liquid compositions can, for example, be prepared by dissolving, dispersing, etc. The active compound is dissolved in or mixed with a pharmaceutically pure solvent such as, for example, water, saline, aqueous dextrose, glycerol, ethanol, and the like, to thereby form the solution or suspension.

If desired, the pharmaceutical composition to be administered may also contain minor amounts of non-toxic auxiliary substances such as wetting or emulsifying agents, pH buffering agents, and other substances such as for example, sodium acetate, triethanolamine oleate, etc.

Those skilled in the art will recognize that the hydrophobic compositions of the instant invention can also be used as an oral or nasal, i.e., mucosal, vaccine having an antigen, to which vaccination is desired, serve as the effector. Such a vaccine can include a composition including a desired antigenic sequence, including, but not limited to, the protective antigen (PA) component of Anthrax, or the Hepatitis B surface antigen (HBs) of Hepatitis B. This composition can then be orally or nasally administered to a subject in need of vaccination.

An “antigen” is a molecule or a portion of a molecule capable of stimulating an immune response, which is additionally capable of inducing an animal or human to produce antibody capable of binding to an epitope of that antigen. An “epitope” is that portion of any molecule capable of being recognized by and bound by a major histocompatability complex (“MHC”) molecule and recognized by a T cell or bound by an antibody. A typical antigen can have one or more than one epitope. The specific recognition indicates that the antigen will react, in a highly selective manner, with its corresponding MHC and T cell, or antibody and not with the multitude of other antibodies that can be evoked by other antigens.

A peptide is “immunologically reactive” with a T cell or antibody when it binds to an MHC and is recognized by a T cell or binds to an antibody due to recognition (or the precise fit) of a specific epitope contained within the peptide. Immunological reactivity can be determined by measuring T cell response in vitro or by antibody binding, more particularly by the kinetics of antibody binding, or by competition in binding using known peptides containing an epitope against which the antibody or T cell response is directed as competitors.

Techniques used to determine whether a peptide is immunologically reactive with a T cell or with an antibody are known in the art. Peptides can be screened for efficacy by in vitro and in vivo assays. Such assays employ immunization of an animal, e.g., a mouse, a rabbit or a primate, with the peptide, and evaluation of the resulting antibody titers.

Also included within the invention are vaccines that can elicit the production of secretory antibodies (IgA) against the corresponding antigen, as such antibodies serve as the first line of defense against a variety of pathogens. Oral or nasal, i.e., mucosal, vaccination, which have the advantage of being non-invasive routes of administration, are the preferred means of immunization for obtaining secretory antibodies, although the vaccination can be administered in a variety of ways, e.g., orally, topically, or parenterally, i.e., subcutaneously, intraperitoneally, by viral infection, intravascularly, etc.

The compositions of the present invention can be administered in oral dosage forms such as tablets, capsules (each including timed release and sustained release formulations), pills, powders, granules, elixirs, tinctures, suspensions, syrups and emulsions.

The dosage regimen utilizing the compounds is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter or arrest the progress of the condition.

Oral dosages of the present invention, when used for the indicated effects, may be provided in the form of scored tablets containing 0.005, 0.01, 0.025, 0.05, 0.1, 0.25, 0.5, 1.0, 2.5, 5.0, 10.0, 15.0, 25.0, 50.0, 100.0, 250.0, 500.0 or 1000.0 mg of active ingredient.

Compounds of the present invention may be administered in a single daily dose, or the total daily dosage may be administered in divided doses of two, three or four times daily. Furthermore, preferred compounds for the present invention can be administered in bucal form via topical use of suitable bucal vehicles, bronchial form via suitable aerosols or inhalants, intranasal form via topical use of suitable intranasal vehicles, or via transdermal routes, using those forms of transdermal skin patches well known to those of ordinary skill in that art. To be administered in the form of a transdermal delivery system, the dosage administration will, of course, be continuous rather than intermittent throughout the dosage regimen. Other preferred topical preparations include creams, ointments, lotions, aerosol sprays and gels, wherein the concentration of active ingredient would range from 0.1% to 50%, w/w or w/v.

The compounds herein described in detail can form the active ingredient, and are typically administered in admixture with suitable pharmaceutical diluents, excipients or carriers (collectively referred to herein as “carrier” materials) suitably selected with respect to the intended form of administration, that is, oral tablets, capsules, elixirs, syrups and the like, and consistent with conventional pharmaceutical practices.

For instance, for oral administration in the form of a tablet or capsule, the active drug component can be combined with an oral, non-toxic pharmaceutically acceptable inert carrier such as ethanol, glycerol, water and the like. Moreover, when desired or necessary, suitable binders, lubricants, protease inhibitors, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, poloxamer, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methylcellulose, agar, bentonite, xanthan gum and the like.

The compounds of the present invention may also be coupled with soluble polymers as targetable drug carriers. Such polymers can include polyvinylpyrrolidone, pyran copolymer, polyhydroxypropyl-methacrylamide-phenol, polyhydroxyethylaspanamidephenol, or polyethyleneoxidepolylysine substituted with palmitoyl residues. Furthermore, the compounds of the present invention may be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels.

Any of the above compositions may contain 0.01-99%, preferably 0.1-10% of the active compounds as active ingredients.

The following EXAMPLES are presented in order to more fully illustrate the preferred embodiments of the invention. These EXAMPLES should in no way be construed as limiting the scope of the invention, as defined by the appended claims.

### EXAMPLES

### Example 1

### Utilization of an Ionic Liquid Forming Cation to Enable the Translocation of Insulin Across an Epithelial Barrier

A composition containing recombinant human insulin and an ionic liquid forming cation, e.g., 1-butyl-3-methylimidazolium chloride, is administered to a test animal, e.g., a mouse. Additional constituents of the composition are specified in Table 1.

TABLE 1
Additional constituents of the composition

Phytic acid
Pluronic F-68
Aprotinin
Solutol HS-15 (SHS)
N-Acetyl Cysteine (NAC)

Administration is done rectally or by injection into an intestinal loop. The experimental procedure involves male BALB/c mice, which are deprived of food, 18 hours prior to the experiment. For intra-intestinal injection the mice are then anesthetized and a 2 cm long incision is made along the center of the abdomen, through the skin and abdominal wall. An intestine loop is gently pulled out through the incision and placed on wet gauze beside the animal. The loop remains intact through the entire procedure and is kept wet during the whole time. The tested compound is injected into the loop, using a 26G needle. For rectal administration the mice are anesthetized and the composition is then rectally administered to the mice, 100 μl/mouse, using a plastic tip covered with a lubricant. To assess penetration, blood glucose levels are subsequently measured.

Blood glucose levels decrease in relation to the amount of insulin absorbed from the intestine into the bloodstream (i.e., in an amount that correlates to the amount of insulin absorbed). Thus, this drug delivery system can replace the need for insulin injections, thereby providing an efficient, safe and convenient route of administration for diabetes patients.

### Example 2

### Utilization of an Ionic Liquid Forming Cation to Enable the Translocation of Heparin Across an Epithelial Barrier

A composition containing heparin and an ionic liquid forming cation, e.g. 1-butyl-3-methylimidazolium chloride, is administered to a test animal, e.g. a mouse. Additional constituents of the composition are specified in Table 1. Administration is done rectally or by injection into an intestinal loop. The experimental procedure involves male BALB/c mice, which are deprived of food, 18 hours prior to the experiment. For intra-intestinal injection the mice are then anesthetized and a 2 cm long incision is made along the center of the abdomen, through the skin and abdominal wall. An intestine loop is gently pulled out through the incision and placed on wet gauze beside the animal. The loop remains intact through the entire procedure and is kept wet during the whole time. The tested compound is injected into the loop, using a 26 G needle. For rectal administration the the mice are anesthetized and the composition is then rectally administered to the mice, 100 μl/mouse, using a plastic tip covered with a lubricant. Partial Thrombin Time (PTT) values are subsequently measured.

Partial Thrombin Time (PTT) values decrease in relation to the amount of heparin absorbed from the intestine loop into the bloodstream (i.e., in an amount that correlates to the amount of heparin absorbed). Therefore, this drug delivery system will replace the use of heparin injections.

### Example 3

### Utilization of an Ionic Liquid Forming Cation for Mucosal Vaccination

The composition for mucosal vaccination contains a desired antigenic sequence, e.g., the PA antigen of Anthrax, and an ionic liquid forming cation, e.g., 1-butyl-3-methylimidazolium. Additional constituents of the pharmaceutical composition are specified in Table 1. Such a composition can be administered to a subject in need of vaccination.

This method allows simple and rapid vaccination of large populations in need thereof. Another advantage of this method is the production of high titers of IgA antibodies and the subsequent presence of IgA antibodies in the epithelial mucosa, which are the sites of exposure to antigens.

Efficacy of vaccination can be demonstrated by the measurement of specific antibody titers, especially for IgA, as well as the measurement of immunological response to stimulation, such as for example, via a cutaneous hypersensitivity reaction in response to subcutaneous administration of antigen.

### OTHER EMBODIMENTS

From the foregoing detailed description of the specific embodiments of the invention, it should be apparent that unique methods of translocation across epithelial and endothelial barriers have been described. Although particular embodiments have been disclosed herein in detail, this has been done by way of example for purposes of illustration only, and is not intended to be limiting with respect to the scope of the appended claims that follow. In particular, it is contemplated by the inventor that various substitutions, alterations, and modifications may be made to the invention without departing from the spirit and scope of the invention as defined by the claims. For instance, the choice of the particular type of tissue, or the particular effector to be translocated is believed to be a matter of routine for a person of ordinary skill in the art with knowledge of the embodiments described herein.

We claim:

1. A composition for the translocation of at least one effector across a biological barrier comprising a therapeutically effective amount of said at least one effector, and a counter ion to the at least one effector.

2. The composition of claim 1, wherein said counter ion is an ionic liquid forming cation.

3. The composition of claim 1 comprising a pharmaceutically acceptable excipient, pharmaceutically acceptable carrier, or a combination thereof.

4. The composition of claim 1, wherein said composition is contained within a capsule.

5. The composition of claim 1, wherein said composition is in the form of a tablet.

6. The composition of claim 1, wherein said composition is enteric-coated.

7. The composition of claim 1, wherein said composition is in the form of an aqueous dispersion.

8. The composition of claim 1, wherein said composition is in the form of a cream.

9. The composition of claim 1, wherein said composition is in the form of an ointment.

10. The composition of claim 1, wherein said composition is in the form of a suppository.

11. The composition of claim 1, wherein said at least one effector is an anionic impermeable molecule.

12. The composition of claim 11, wherein said anionic impermeable molecule is a polysaccharide.

13. The composition of claim 12, wherein said polysaccharide is a glycosaminoglycan.

14. The composition of claim 13, wherein said glycosaminoglycan is selected from the group consisting of: heparin; heparan sulfate; chondroitin sulfate; dermatan sulfate; hyaluronic acid; and pharmaceutically acceptable salts thereof.

15. The composition of claim 11, wherein said anionic impermeable molecule is a bioactive molecule.

16. The composition of claim 15, wherein said bioactive molecule is selected from the group consisting of: insulin; erythropoietin (EPO); glucagon-like peptide 1 (GLP-1); αMSH; parathyroid hormone (PTH); growth hormone; calcitonin; interleukin-2 (IL-2); α1-antitrypsin; granulocyte/monocyte colony stimulating factor (GM-CSF); granulocyte colony stimulating factor (G-CSF); T20; anti-TNF antibodies; interferon α; interferon β; interferon γ; lutenizing hormone (LH); follicle-stimulating hormone (FSH); enkephalin; dalargin; kyotorphin; basic fibroblast growth factor (bFGF); hirudin; hirulog; lutenizing hormone releasing hormone (LHRH) analog; brain-derived natriuretic peptide (BNP); and neurotrophic factors.

17. The composition of claim 1, wherein said at least one effector is a pharmaceutically active agent.

18. The composition of claim 17, wherein said pharmaceutically active agent is selected from the group consisting of: a hormone; a growth factor; a neurotrophic factor; an anticoagulant; a bioactive molecule; a toxin; an antibiotic; an anti-fungal agent; an antipathogenic agent; an antigen; an antibody; an antibody fragment; an immunomodulator; a vitamin; an antineoplastic agent; an enzyme; and a therapeutic agent.

19. The composition of claim 1, wherein said at least one effector is a nucleic acid or a nucleic acid mimetic.

20. The composition of claim 19, wherein the nucleic acid is a DNA or DNA-mimetic.
